(19) United States
(12) Patent Application Publication
(10) Pub. No.: US 2012/0295336 A1
(43) Pub. Date: Nov. 22, 2012

(54) Microalgae Cultivation System for Cold Climate Conditions

(76) Inventors: David A. Hazlebeck, El Cajon, CA (US); Mitch Zafer, San Diego, CA (US); William S. Rickman, Lebanon, TN (US)

(21) Appl. No.: 13/112,740

(22) Filed: May 20, 2011

(51) Int. Cl. C12N 1/12; C12M 1/02

(52) U.S. Cl. 435/257.1; 435/289.1; 435/292.1; 435/293.1

(57) ABSTRACT

A system and method are provided for growing microalgae in cold climate areas. The system includes an expanding Plug Flow Reactor with a plurality of ponds used to grow algae by mixing a culture fluid with a nutrient. To minimize the loss of heat due to environmental factors, the expanding Plug Flow Reactor is covered with a translucent, light-transmitting cover and is lined with an insulation liner. In addition, an underground sump and pump are provided and connected to the expanding Plug Flow Reactor. The sump is provided to store the algae at night when ambient air temperature is at its coldest. An adjacent power plant provides: (1) heat byproducts to warm the culture and (2) CO₂ for use as a source of carbon in photosynthesis.

### FIELD OF THE INVENTION

The present invention pertains generally to systems and methods for growing microalgae. More particularly, the present invention pertains to the use of a system that can grow microalgae in a cold climate area. The present invention is particularly, but not exclusively, useful as a system for growing algae in a cold climate area that uses heat byproducts from power plants, and an underground sump, to maintain a temperature conducive to algae growth.

### BACKGROUND OF THE INVENTION

As worldwide petroleum deposits decrease, there is rising concern over petroleum shortages and the costs that are associated with the production of carbon-based fuel sources. As a result, alternatives to products that are currently processed from petroleum are being investigated. In this effort, biofuel has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats are reacted with an alcohol, such as methanol.

Apart from using animal fats, the creation of biofuels from plant oils has gained wide attention in recent years. The process of creating biofuel from plant oils, of course, necessarily begins by growing and harvesting plants such as algae cells. In particular, algae is known to be one of the most efficient plants for converting solar energy into cell growth, so it is of particular interest as a biofuel source.

In an algae cultivation system, the algae cells are typically grown as part of a liquid medium that is often exposed to sunlight to promote photosynthetic growth. Further, the algae cell growth process normally requires the liquid medium to be circulated through the system. Due to heating requirements for cell growth, geographic areas with warmer climates and higher degrees of solar insolation are preferred locations for algae cultivation systems. In particular, locations with warmer climates allow the temperature of the liquid culture to remain sufficiently warm, for a sufficient period of time, to promote efficient algae cell growth. On the other hand, freezing or near-freezing conditions will cause serious algae cell growth problems. Cold temperatures will greatly inhibit, or even stop, the growth of algae cells. Clearly, slowing or stopping the growth of algae cells is detrimental to an algae cultivation system. And, to produce biofuel in a cost effective manner as compared to carbon-based fuel products, disruptions in algae cultivation cannot occur. Consequently, any stopping or slowing of algae growth will make an algae growth system economically unsustainable.

Like most plants, algae cells do not grow effectively in cold weather. At the present time, the predominant methods used to grow algae for use in biofuel production are limited to geographic areas with warmer climates. As a consequence, many suitable sites in cold climate areas are not being efficiently exploited. Thus, by developing an algae growth system that is optimized for cold climate regions, the geographic footprint available for biofuel production facilities could be increased dramatically.

In light of the above, it is an object of the present invention to provide a system and method for growing microalgae for biofuel production in cold climate areas. Another object of the present invention is to provide a system and method for growing microalgae that expands the geographical footprint of areas suitable for biofuel production. Still another object of the present invention is to mitigate pollution by recycling heat and CO₂ byproducts produced by power plants to grow microalgae. Yet another object of the present invention is to provide a system and method for growing microalgae for biofuel production in cold climate areas that is simple to implement, easy to use, and comparatively cost effective.

### SUMMARY OF THE INVENTION

In accordance with the present invention, a system and method for cold climate algae growth is provided. As envisioned for the present invention, the system is constructed in a cold climate area and is co-located with a power plant that produces heated cooling water and CO₂ as byproducts. Structurally, the system comprises an expanding Plug Flow Reactor (ePFR) connected to an underground sump. The underground sump is provided for storing the algal culture during periods of extreme cold temperature. In an operation of the present invention, the algal culture can be transferred from the ePFR to the sump, and vice versa, as required to ensure algae growth is not hindered by cold temperatures.

As mentioned above, the system of the present invention begins with a plug flow reactor (PFR) that is used to grow an algal culture. In further detail, the PFR comprises a plurality of individual ponds. Preferably, the ponds are each elongated in shape and form a raceway type cultivation pond with a configuration that is well-known in the trade. Collectively, the plurality of individual ponds creates an expanding PFR (ePFR), meaning that the ponds are arranged in order of increasing capacity, with the first pond being the smallest and kept under sterile conditions. Importantly, each pond is in fluid communication with adjacent ponds to facilitate transfer from one pond to the next larger pond as required.

For their construction, each pond of the ePFR is preferably constructed with a sloped bottom portion that provides for gravitational fluid flow through the pond to facilitate the mixing of algae cells with nutrients. Furthermore, the bottom portion is positioned between opposite sidewalls to form a shallow fluid flow channel that will maximize the exposure of the algae to sunlight. A light-transmitting, insulating cover can be attached to each pond to extend between the sidewalls, and the cover is positioned opposite the bottom of the pond. Further, the light-transmitting cover should be transparent or translucent, and constructed with lightweight plastic to allow for floatation on top of the algal culture. To further promote floatation, the plastic used to construct the cover may include sealed air cells. By constructing the cover in this manner, the cover is dual-purpose as solar energy required by the algae cells for photosynthesis can still enter the system, and the cover provides an insulative effect. In addition to the light-transmitting cover, an insulation liner is constructed on top of the bottom and the sidewalls of each pond. For a preferred embodiment of the present invention, the insulation liner is sprayed onto the bottom and sidewalls during construction of the ePFR to prevent heat losses due to thermal conduction to the ground.

In addition to the ePFR, the present invention includes an underground sump that is connected by a pipe to the ePFR. In one embodiment, the underground sump may be divided into separate chambers, with each chamber receiving algal culture from a dedicated cultivation pond of the ePFR. In an alternate configuration, one underground sump may be provided for each of the individual cultivation ponds. As contemplated for the present invention, the underground sump is connected to the downstream end of the ePFR by a pipe having a valve. In this configuration, the valve can be opened to allow for gravity flow of the algal culture from the ePFR during periods of extreme cold temperature. Most often, these periods of extreme cold temperature occur at night. In a preferred embodiment, only one pipe is used to move the algal culture into the sump and from the sump back into the ePFR. Configurations using multiple pipes, however, may also be used. While gravity flow may be sufficient to move the algal culture from the ePFR to the sump, a pump is necessary to transfer the algal culture from the sump back to the upstream end of the ePFR. Furthermore, the pump may also be configured to move the algal culture from the ePFR to the sump, if necessary.

The system of the present invention also adds heat from the power plant to the underground sump. To do this, the power plant is connected to the underground sump by a water pipe. This water pipe carries heated cooling water from the power plant to a first heat exchanger placed in the underground sump. Once the heated cooling water reaches the first heat exchanger, the heat from the heated cooling water is transferred into the stored algal culture in the underground sump. To facilitate the addition of heat to the growing algal culture, a second heat exchanger is provided and placed into the ePFR. The water pipe is constructed with a directional valve that can close to stop the flow of heated cooling water. And, the directional valve can be configured to direct the heated cooling water into either the first heat exchanger or the second heat exchanger. When heat is required in the ePFR, heated cooling water is directed to the second heat exchanger which will transfer heat from the heated cooling water into the culture in the ePFR. The cooled cooling water effluent from the heat exchanger flows back to the power plant.

Flue gas produced by the power plant is recycled into the system of the present invention. Once the flue gas leaves the power plant, it is piped to a CO₂ absorber through a gas pipe. Makeup media is also piped from an algae processor to the CO₂ absorber. The makeup media is created in the algae processor by separating and removing mature algae cells from the algal culture and has a high concentration of sodium carbonate. This makeup media will act as an absorbent for CO₂ and heat present in the flue gas. Once absorption has occurred, makeup media is enriched with bicarbonate. At this point, the makeup media is added to the ePFR to act as a heat and carbon source for the growing algal culture.

In operation, the light-transmitting, transparent/translucent insulating cover is attached between the sidewalls of the ePFR. This attachment can occur prior to the introduction of algal culture into the ePFR or after the introduction of algal culture into the ePFR. Both heat losses and evaporation losses are minimized by placing the cover onto the ePFR. When the algal culture is introduced into the ePFR, it remains in continuous motion due to: (1) the sloped configuration of the individual ponds of the ePFR and (2) a mixing means, such as a paddle or a pump. While the algal culture is being mixed within the ePFR, byproducts from the power plant are being collected. As mentioned previously, these byproducts are heated cooling water and flue gas. The heated cooling water is piped directly from the power plant through the second heat exchanger and into the ePFR to provide heat to the growing algal culture. In addition, flue gas from the power plant is piped to the CO₂ absorber where it is absorbed by makeup media. After absorption, the makeup media is fed into the ePFR through a conduit to both nourish and heat the growing algal culture.

During periods of extreme cold temperature, the valve of the underground sump is opened to allow for the algal culture to flow from the ePFR into the underground sump. While stored in the underground sump, the algal culture will be protected from the type of growth disruptions that may be caused by cold weather. This is accomplished in several ways: (1) heated cooling water from the power plant is piped to the underground sump via the first heat exchanger to warm the stored algal culture, (2) heat losses due to environmental conditions are minimized by the insulative properties of the surrounding soil, and (3) the surface area of the algal culture is reduced when exposed to ambient air. Once the period of extreme cold temperature has passed, the algal culture is pumped from the underground sump back to the ePFR where algae cells can continue to grow.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a diagram of the layout of the system for the present invention in an operational environment;

FIG. 2 is a schematic diagram of fluid flow through the system of the present invention;

FIG. 3 is a cross-section view of the fluid flow channel as seen along the line 3-3 in FIG. 1; and

FIG. 4 is a top-view of an expanding Plug Flow Reactor (ePFR) having four cultivation ponds.

### DESCRIPTION OF THE PREFERRED EMBODIMENTS

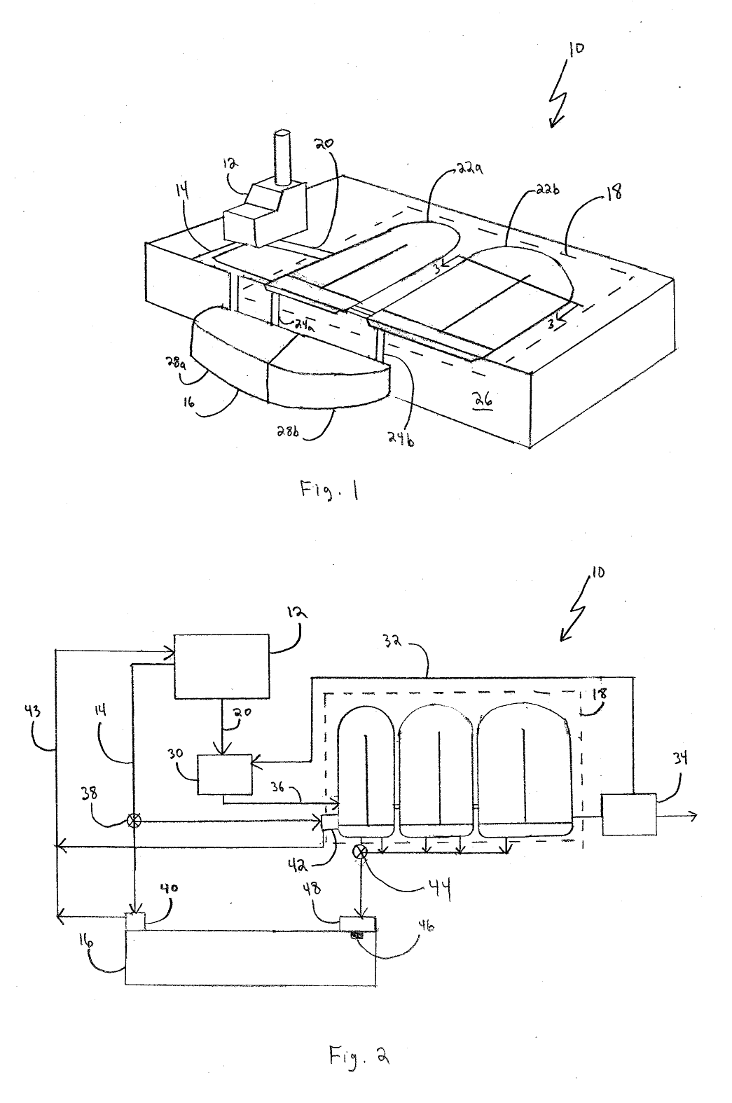

Referring initially to FIG. 1, the system of the present invention is shown in an operational environment and generally designated 10. As shown, the system 10 is built on a generally flat site and incorporates a conventional power plant 12. In the system, the power plant 12 is connected to a water pipe 14 for carrying heated cooling water from the power plant 12 to both an underground sump 16 and an expanding Plug Flow Reactor (ePFR) 18. In addition, a gas pipe 20 is connected to the power plant 12 to provide flue gas to the system 10. As shown in FIG. 1, an exemplary configuration of the ePFR 18 comprises two cultivation ponds 22a-b. Each cultivation pond 22a-b is constructed with a transfer pipe 24a-b that connects a respective cultivation pond 22a-b with the underground sump 16. The underground sump 16 is surrounded by soil 26 and includes partitions 28a-b that allow the algal culture from each cultivation pond 22a-b to be stored separately when required. In an alternate configuration, the underground sump 16 may be built without partitions when the algal culture from the cultivation ponds 22a-b can be mixed for storage in the underground sump 16.

Referring now to FIG. 2, a schematic layout for fluid flow through the system 10 is shown. In operation, flue gas is produced by the power plant 12 and travels through the gas pipe 20 to the CO₂ absorber 30. The CO₂ absorber 30 is also connected to a conduit 32 that provides makeup media created at an algae processor 34. The primary purpose of the makeup media is to absorb CO₂ from the flue gas. Once this absorption takes place, the makeup media becomes heated and enriched with CO₂. At this point, the makeup media is added to the ePFR 18 through at least one injector pipe 36 to heat and nourish the growing algal culture.

Referring again to FIG. 2, the fluid flow path of heated makeup water produced by the power plant 12 is also shown. Upon leaving the power plant 12, the heated cooling water travels through a water pipe 14 containing a directional valve 38. By controlling the operation of the directional valve 38, the heated cooling water can be directed to: (1) the underground sump 16 via a first heat exchanger 40 or (2) the ePFR 18 via a second heat exchanger 42. Or, the directional valve 38 can be closed to stop the flow of heated cooling water to the system 10. In both the underground sump 16 and the ePFR 18, the heated cooling water will be used to modulate the temperature of the algal culture. A cooling water return line 43 is also provided to return cooled cooling water effluent back to the power plant 12 from the first heat exchanger 40 and the second heat exchanger 42.

Still referring to FIG. 2, the flow of algal culture between the ePFR 18 and the underground sump 16 is also illustrated. During periods of extreme cold temperature (e.g. at night or during a blizzard), algal culture in the ePFR 18 is moved to the underground sump 16. To accomplish this, a gate valve 44 is opened to allow the algal culture to flow into the underground sump 16 through an inlet/outlet 46. Once the period of extreme cold temperature has passed, the algal culture is pumped back into the ePFR 18 using a pump 48 that is connected to the underground sump 16.

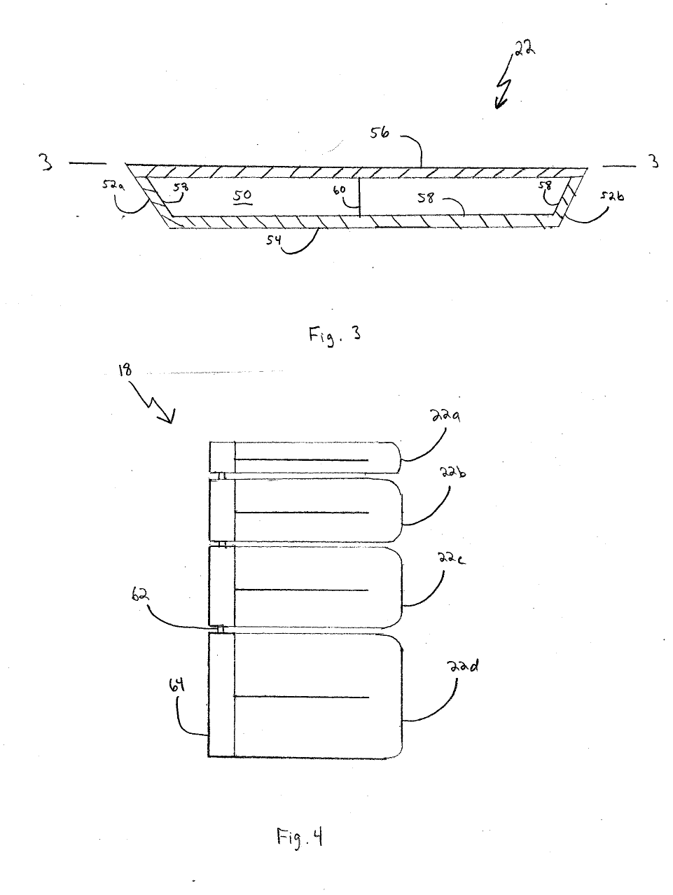

Now referring to FIG. 3, a cross-section of a cultivation pond 22 of the ePFR 18 is shown as seen along the line 3-3 in FIG. 1. As depicted, the cultivation pond 22 has a shallow fluid flow channel 50 that is formed by two side walls 52a, 52b of the ePFR 18 and a bottom portion 54. It should be noted that the trapezoidal shape of the fluid flow channel 50 shown in FIG. 3 is for illustrative purposes only as the fluid flow channel 50 may take any shape suitable for the operation of a cultivation pond 22. On top of the fluid flow channel 50 is a translucent or transparent cover 56 that extends from sidewall 52a to sidewall 52b and is parallel to the bottom portion 54 of the ePFR 18. A further safeguard against convection losses from the cultivation pond 22 is the use of an insulation liner 58 that that is sprayed onto both sidewalls 52a-b and the bottom portion 54 of the ePFR 18 during construction or at any other time when the cultivation pond 22 is empty. Also, a divider 60 is provided to promote the type of circular flow most conducive to algae growth in the cultivation pond 22.

Now referring to FIG. 4, an ePFR 18 having four cultivation ponds 22a-d is shown. It should be noted that four cultivation ponds 22a-d are being used for exemplary purposes as any number of cultivation ponds 22 may be used for the system 10. As illustrated, the four cultivation ponds 22a-d of the ePFR 18 are arranged in order of increasing capacity with algal culture growth beginning in the smallest cultivation bond 22a. Each cultivation pond 22a-d contains similar structural components as labeled for cultivation pond 22d. To transfer fluid to an adjacent cultivation pond 22a-d, a connecting pipe 62 is provided. In addition, the cultivation pond 22d is built with a housing 64 that may house a mixing device or any other hardware associated with operating the cultivation pond 22d. In addition to the divider 62, a mixing device (not shown) will also promote circular flow of the algal culture.

While the particular Microalgae Cultivation System for Cold Climate Conditions as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A Cold Climate Algae (CCA) system which comprises:
a plug flow reactor having a cultivation pond with a first end and a second end, wherein an algal culture, with media, is introduced into the pond at the first end thereof to grow algae cells therein;
a power plant for generating flue gas;
an algae processor connected to the second end of the pond to separate and remove mature algae cells from the media to create a makeup media;
a CO₂ absorber for receiving makeup media from the algae processor and for receiving flue gas from the power plant, wherein the flue gas heats the makeup media and enriches the makeup media with CO₂ prior to returning the heated and enriched makeup media to the pond for mixing with the algal culture in the pond; and
an underground sump connected in fluid communication with the cultivation pond for selectively transferring the algal culture therebetween.

2. A CCA system as recited in claim 1 wherein the power plant generates heated cooling water and the system further comprises a means for selectively transferring heat from the heated cooling water from the power plant to algal culture in the cultivation pond and algal culture in the underground sump.

3. A CCA system as recited in claim 1 wherein the Plug Flow Reactor is an expanding plug flow reactor (ePFR) comprising a plurality of cultivation ponds arranged in order of increasing capacity, wherein each cultivation pond of the plurality is in fluid communication with the sump, and the individual cultivation ponds have a unique fluid capacity.

4. A CCA system as recited in claim 1 further comprising a pump connected in fluid communication with the first end of the cultivation pond and the underground sump for selectively pumping the algal culture between the underground sump and into the cultivation pond.

5. A CCA system as recited in claim 2 further comprising:
a first heat exchanger connected to the underground sump, wherein the first heat exchanger transfers heat from the heated cooling water to the underground sump to warm the algal culture in the underground sump; and
a second heat exchanger connected to the ePFR, wherein the second heat exchanger transfers heat from the heated cooling water to the ePFR to warm the algal culture in the ePFR.

6. A CCA system as recited in claim 1 wherein the cultivation pond has a bottom portion extending from the first end of the cultivation pond to the second end of the cultivation pond with a bottom portion positioned between opposed sidewalls, and wherein the bottom portion is sloped to cause the algal culture to flow from the first end to the second end of the cultivation pond.

7. A CCA system as recited in claim 6 wherein the system further comprises:
a light-transmitting cover extending between the sidewalls opposite the bottom of the cultivation pond; and
an insulation liner affixed onto the bottom and the sidewalls of the cultivation pond.

8. A CCA system as recited in claim 7 wherein the light-transmitting cover is constructed with a material selected from a group comprising a translucent material and a transparent material.

9. A CCA system as recited in claim 8 wherein the selected material is lightweight plastic with sealed air cells to promote floatation and insulation.

10. A method for growing microalgae in a cold climate which comprises the steps of:
introducing an algal culture, with media, into a cultivation pond of a plug flow reactor, wherein the cultivation pond has a first end and a second end, wherein the algal culture is introduced into the pond at the first end thereof to grow algae cells therein;
generating flue gas with a power plant;
separating mature algae cells from the media to create a makeup media;
heating and enriching the makeup media with the flue gas;
returning the heated and enriched makeup media to the first end of the pond for mixing with algal culture in the pond; and
selectively transferring the algal culture from the cultivation pond into an underground sump, wherein the algal culture is transferred to the underground sump during a period of extreme cold temperature.

11. A method as recited in claim 10 further comprising the steps of:
generating heated cooling water with the power plant; and
selectively transferring heat from the heated cooling water from the power plant to the algal culture in the cultivation pond and the algal culture in the underground sump.

12. A method as recited in claim 11 further comprising the steps of:
transferring heat from the heated cooling water to the underground sump via a first heat exchanger to warm the algal culture in the underground sump; and
transferring heat from the heated cooling water to the ePFR via a second heat exchanger to warm the algal culture in the ePFR.

13. A method as recited in claim 10 wherein a plurality of cultivation ponds is provided, and wherein the plurality of cultivation ponds comprise an expanding Plug Flow Reactor, and wherein the plurality of cultivation ponds are arranged in order of increasing capacity.

14. A method as recited in claim 11 wherein the cultivation pond has a bottom portion extending from the first end of the cultivation pond to the second end of the cultivation pond and positioned between opposed sidewalls, and wherein the bottom portion is sloped to cause the algal culture to flow from the first end to the second end of the cultivation pond.

15. A method as recited in claim 10 further comprising the steps of:
insulating the algal culture with an insulation liner, wherein the insulation liner is affixed onto the bottom and the sidewalls of the cultivation pond; and
attaching a light-transmitting cover to the cultivation pond, wherein the light-transmitting cover extends between the sidewalls opposite the bottom of the cultivation pond.

16. A method as recited in claim 15 wherein the light-transmitting cover is constructed with a material selected from a group comprising a transparent material and translucent material, and wherein the selected material is a lightweight plastic with sealed air cells to promote floatation.

17. A Cold Climate Algae (CCA) system which comprises:
a means for cultivating an algal culture having a first end and a second end, and wherein the algal culture has a media and is introduced into the cultivating means at the first end;
a means for generating flue gas;
a means for separating and removing mature algae cells from the media to create a makeup media;
a means for receiving makeup media from the separating and removing means and for receiving flue gas from the generating means, wherein the flue gas heats the makeup media and enriches the makeup media with CO₂ prior to returning the heated and enriched makeup media to the first end of the cultivating means for mixing with the algal culture; and
a means for storing the algal culture during periods of extreme cold, wherein the storing means is connected in fluid communication with the cultivating means for selectively transferring the algal culture therebetween.

18. A CCA system as recited in claim 17 wherein the means for cultivating the algal culture is an expanding Plug Flow Reactor, wherein the expanding Plug Flow Reactor comprises a plurality of cultivation ponds, and wherein each cultivation pond has a bottom portion extending from the first end of the cultivation pond to the second end of the cultivation pond and is positioned between opposed sidewalls.

19. A CCA system as recited in claim 18 further comprising:
a means for insulating the algal culture, wherein the means for insulating the algal culture is affixed onto the bottom portion and the sidewalls of the means for cultivating an algal culture; and
a means for covering the means for cultivating an algal culture to prevent losses due to heat and evaporation.

20. A CCA system as recited in claim 17 wherein the generating means generates heated cooling water, and wherein the heated cooling water is transferred from the generating means to the algal culture in the cultivating means and to the algal culture in the storage means.
